(19) United States
(12) Patent Application Publication
(10) Pub. No.: US 2023/0136583 A1
(43) Pub. Date: May 4, 2023

(54) SYSTEMS AND METHODS FOR MODELING NUTRITIONAL INTAKE DATA

(71) Applicant: Fig Health, Inc. d/b/a Culina Health, Hoboken, NJ (US)

(72) Inventors: Vyacheslav Kuyan, Glen Ridge, NJ (US); Marta Moreno, Salem, MA (US)

(21) Appl. No.: 17/981,144

(22) Filed: Nov. 4, 2022

Related U.S. Application Data
(60) Provisional application No. 63/263,530, filed on Nov. 4, 2021.

(51) Int. Cl. G16H 20/60

(52) U.S. Cl. CPC G16H 20/60

(57) ABSTRACT

A nutritional intake modeling (NIM) computing device provides access to a nutrition platform to a plurality of patients via their devices. The computing device is configured to: (1) receive, from patients, data input captured by the patient's respective devices, (2) generate, using a predictive model, an intake item based on a profile patient and the received data input, (3) add the intake item to the patient's profile, and (4) store the patient's profile on a storage device communicatively coupled to the NIM computing device for subsequent retrieval. Training datasets are validated by trusted users, such as dietitians. Varying modules are used to provide real-time intervention for a patient based on current goals and captured data inputs.

### CROSS-REFERENCE TO RELATED APPLICATIONS

This application claims the benefit of U.S. Provisional 63/263,530, filed Nov. 4, 2021, which is hereby incorporated by reference as if submitted in its entirety.

### TECHNICAL FIELD

Exemplary embodiments of the present invention relate generally to artificial intelligence systems and methods for predictive modeling and, more specifically, to machine learning systems and methods for predictive modeling related to nutritional intake, tracking, and logging.

### BACKGROUND OF THE INVENTION

Nutritional intake is a critical factor in wellness and overall health but is notoriously difficult to capture and catalog. A complete picture of a person's nutritional intake can provide a wealth of knowledge regarding health and wellness improvements, diagnoses, and more.

Detailed and complete records are extremely difficult to compile outside of a 24-hour managed care setting, and most attempts at comprehensive nutritional intake tracking result in inaccurate and incomplete data. In most cases, compiling detailed records is impractical and does not extend to the daily habits of the average person.

With the ubiquity of mobile computing devices, there are known methods of allowing a user to log their nutritional intake by manual entry. Existing applications and technologies seek to make this process more effective by using better interfaces and improved note-taking processes, but still struggle with prolonged user engagement. And despite these conveniences, it has been found that the novelty of new applications and features wears off quickly, leading to incomplete nutritional intake records.

Incomplete nutritional intake data can greatly reduce the usefulness of logging such data in the first place. Unreliable and incomplete nutritional datasets cannot be employed to effectively recommend improvements to lifestyle and eating habits, diagnose problems, or forecast health- and wellness-related issues in the future based on current trends.

It is therefore an unmet need in the prior art for systems and methods to aid in improving the collection and compiling comprehensive, complete, and accurate nutritional intake logs.

### BRIEF SUMMARY OF THE INVENTION

Exemplary embodiments of the present disclosure pertain to systems and methods by which mobile data (location, search history, acceleration, app data, etc.), environmental factors, nutritional database, user generated data and behavior, along with images of food items can be integrated into a single model to predict a personalized nutritional plan and to interpolate missing data signals in nutritional intake profiles.

An object of the present invention to promote adherence to a nutrition plan and allow for tracking nutritional intake when direct data (e.g., logging) is not user provided, or is incomplete.

In one aspect, a nutritional intake modeling (NIM) computing device including at least one processor in communication with a memory device may be provided. At least one processor may be configured to: (i) provide access to a nutrition platform to a plurality of user devices associated with the plurality of users, (ii) receive, from at least one user of the plurality of users, data input captured by the at least one user's user device, generate, using a predictive model, an intake item based on a profile of the at least one user and the received data input, (iii) add the intake item to the user's profile, and (iv) store the user's profile on a storage device communicatively coupled to the NIM computing device. The NIM computing device may include additional, less, or alternate functionality, including that discussed elsewhere herein.

In another aspect, a non-transitory computer readable medium may be provided. The non-transitory computer readable medium may be configured to store instructions that when executed by a processor, implement: (i) creating a predictive model using a plurality of training datasets, (ii) providing access to a nutrition platform to a plurality of user devices associated with a plurality of users, (iii) providing access to the nutrition platform to a plurality of provider devices associated with a plurality of providers, (iv) receiving, from a user of the plurality of users, data input captured by the user's device, (v) generate, using the predictive model, an intake item based on a profile of the user and the received data input, (vi) add the intake item to the user's profile, and (vii) store the user's profile on a storage device for subsequent retrieval. The non-transitory computer readable medium may include additional, less, or alternate functionality, including that discussed elsewhere herein.

In yet another aspect, a computer-based method may be provided. The method comprising (i) receiving data input captured by a client device associated with a patient, (ii) generating, using a predictive model, an intake item based on the received data input, (iii) updating a record associated with the patient to include the intake item, (iv) generating an alert based at least in part on at least one parameter stored in the record and the intake item, and (v) sending the alert to the client device. The computer-based method may include additional, less, or alternate actions, including those discussed elsewhere herein.

It is an object of this invention to provide predictive modeling systems and methods of the type generally described herein, being adapted for the purposes set forth herein, and overcoming disadvantages found in the prior art. These and other advantages are provided by the invention described and shown in more detail below.

### BRIEF DESCRIPTION OF THE SEVERAL VIEWS OF THE DRAWINGS

Novel features and advantages of the present invention, in addition to those mentioned above, will become apparent to those skilled in the art from a reading of the following detailed description in conjunction with the accompanying drawings wherein identical reference characters refer to identical parts and in which:

FIG. 1 is a schematic diagram of an exemplary training embodiment of predictive model;

FIG. 2 is a schematic diagram of an exemplary training embodiment of a predictive model;

FIG. 3 is a schematic diagram of an exemplary method of using the predictive model;

FIG. 4 is an exemplary computing device in accordance with one or more of the disclosed embodiments;

FIG. 5 depicts an exemplary flow diagram 500 in accordance with at least one embodiment of the disclosure; and

FIG. 6 depicts an exemplary flow diagram 600 in accordance with at least one embodiment of the disclosure.

### DETAILED DESCRIPTION OF THE INVENTION

Exemplary embodiments of the present invention are directed at the integration of all associated data items, including user generated and previously generated items (e.g., cataloged food items with associated nutritional information, locations of restaurants and their associated menu items). This data can originate from main sources, and though each source may not be as accurate (at single calorie resolutions) as manual entry, their integration and compounding through a multi-level artificial intelligence (AI) model (CNN, RNN, LSTM) that has been trained on accurate gold-standard data can produce accurate nutritional predictions without changing user behavior. This approach maximizes the effectiveness of passive data capture and offers integrated prediction that can be performed on the backend of mobile devices (computation can also be performed on cloud, edge devices, tablets or other microchip-based systems). This approach also allows for whole-period data capture wherein gaps in the data fields (missing images or food logs) are interpolated through the results of the data model. The calculated data can be provided to the user through a mobile application (accessed directly or through notifications) or through haptic feedback on a device or an associated gadget.

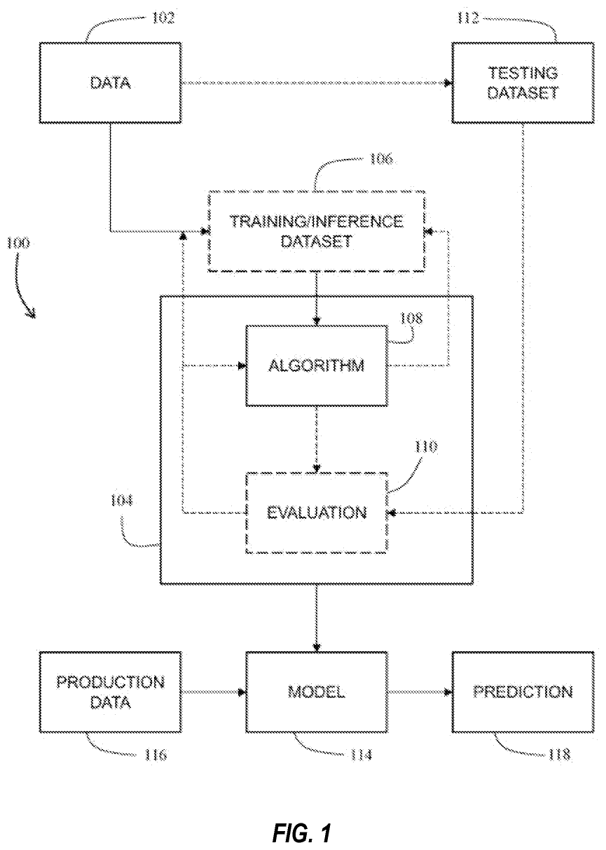

In some embodiments, the systems can include one or more computer programs on one or more computers in one or more locations, configured for use in a method as described herein. Turning to FIG. 1, a schematic diagram is shown illustrating an exemplary embodiment of general implementation elements of the disclosed nutritional predictive modeling system 100. Data 102 are generally provided for use by a machine learning (ML) controller 104 for analysis to build a correlation between several inputs, which may include mobile data, user specific data, nutritional data and geolocation data. At least one training dataset 106 is prepared from the data 102 according to the requirements of the at least one algorithm 108 or machine learning construct chosen during the implementation of the disclosed system 100.

As understood by those of skill in the art, AI-based classification techniques can vary depending on the desired implementation, without departing from the disclosed technology. For example, AI classification schemes can utilize one or more of the following, alone or in combination: hidden Markov models; recurrent neural networks (RNNs); convolutional neural networks (CNNs); deep learning; Bayesian symbolic methods; general adversarial networks (GANs); support vector machines (SVMs); image registration methods; or applicable rule-based system. Where regression algorithms are used, they may include but are not limited to a Stochastic Gradient Descent Regressor, a Passive Aggressive Regressor, or other such algorithms. Self-taught methods, which have been previously trained on similar data types but originating from a different domain (or the same domain through simulated means or different geographical territories), can also be employed.

ML classification models can also be based on clustering algorithms (e.g., a mini-batch K-means clustering algorithm), a recommendation algorithm (e.g., a min-wise hashing algorithm or Euclidean locality-sensitive hashing (LSH) algorithm), or an anomaly detection algorithm, such as a local outlier factor. Additionally, machine learning models can employ a dimensionality reduction approach, such as, one or more of: a mini-batch dictionary learning algorithm, an incremental principal component analysis (PCA) algorithm, a latent Dirichlet allocation (LDA) algorithm, a mini-batch K-means algorithm, or other such comparable means. Model training can take the form of supervised or unsupervised in the integration of the various datasets described herein.

In some implementations, multiple different types of AI training models may be deployed in both the ML training algorithm(s) 108 and in an evaluation module 110. By way of example, general forms of machine learning can be used in order to dynamically adjust classifications and predictions and second- and third-order correlations related thereto and between the datasets used. As recognized by those of skill in the art, the selected AI model or models do not simply contain categorization instructions, but is/are a way to provide feedback on and improve nutritional intake characterization, and prediction operations on subject populations and their environments. In some embodiments, the different AI models discussed herein can be deployed in a specific order to achieve dynamic feedback and iterative model improvements through repeat cycles and model tuning to optimize nutritional intake characterization based on training datasets 106 or optional testing datasets 112 (if available), and to further optimize useful correlative results stemming from those categorizations. First, CNNs can be used in the nutritional characterization process to classify relevant correlations in food image data, location data, nutrition data, behavior data, restaurant data and the like. Second, reinforced learning (RL) and RL agents can be used and rewarded for achieving desired outcomes, both from the CNN classifications and for predefined desirable outcomes, such as known food image classifications. The RL agents can be supervised or unsupervised. Third, GANs can be used to choose between conflicting RL agents. GANs can involve minimal human supervision, relying on humans only for selecting which RL agents to input as nodes to the GANs. Fourth, RNNs can take the winning RLs as input nodes to create a feedback and feed-forward system, so that learning can be continuous and unsupervised.

In some embodiments, the preparation of a training dataset 106 is optional. For example, such high-quality data 102 may be provided that pre-processing needs are low or nonexistent. In some embodiments, unsupervised ML models may also be employed at 108 whereby extensive categorization, labeling and other such pre-processing activities are not necessary.

The machine learning controller 104 could be embodied as, for instance, a convolutional neural network (CNN), reinforcement learning model (RL), or another numeral method model. The deep learning controller is trained at 104 to build one or more models 114 that can be used to estimate nutritional intake, classify food images and correlate the same with nutritional intake predictions, identify food items most likely to be consumed at a particular location, and to interpolate records for incomplete nutritional intake logs. With the initial correlations formed for the training group, additional prediction and group classifications can be made for future inference groups, whether through new training datasets 106 or verifiable testing datasets 112. This allows that, for a given production dataset 116, the model 114 can predict and approximate 118 such things as estimated nutritional intake, identify food items and assign nutritional values to them, predict food items likely to be consumed at a location, and interpolate missing records for incomplete nutritional intake logs, for instance.

Additional test data comprising verifiable testing datasets 112 paired with known nutritional profiles may be passed through the model 114 to be evaluated at 110. In some embodiments, the evaluation step 110 can include the adjustment of one or more model weight parameters, inner or outer, for overall improvement of model confidence prediction. In some embodiments, the input data and features and labels of individual datasets 106 can be adjusted based on the feedback. Therefore, some exemplary embodiments are provided with a mechanism for adjusting the relative weights assigned to input variables by the model function being constructed, as at 110.

Once verified, the model 114 can be passed real production data 116 to obtain result predictions and nutritional profiles 118. The model 114 can be trained until a minimum threshold of confidence is reached. In some embodiments, the model 114 is continuously trained with the addition of new data and examples. In additional embodiments, the training and inference datasets 106 can be the same, different, or subgroups of each other.

In some embodiments, a CNN is provided to train a deep learning controller 104. The CNN can contain as many layers as desired or required and may include hidden layers. The CNN can be implemented with optionally variable tuning and optimization parameters. The CNN model can be trained for as many cycles as needed, such as until the confidence for a specific value set is reached. The confidence score can be set by the expert user, through regression of the integrated models or the existing minimum threshold that is required by actuarial standards. By way of example, the model can be trained to give a sufficient prediction based on input A until a 90% confidence is reached.

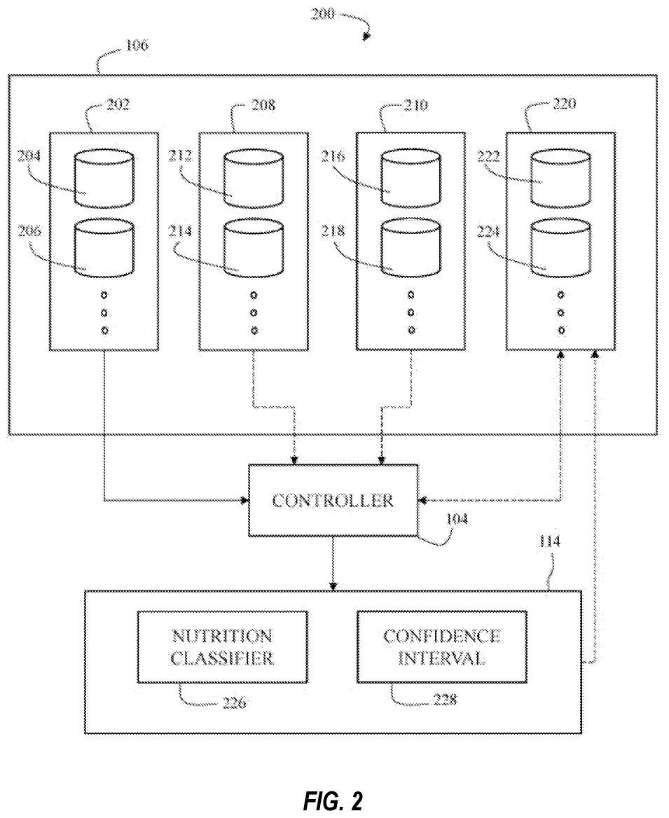

Turning to FIG. 2, an exemplary model training embodiment of the machine learning controller 104 is depicted in schematic form by system 200. The training dataset 106 is prepared with at least one mobile data training dataset 202 from, for instance, mobile smartphone location data 204. These data can be sourced from one or more user devices as represented by an additional data source 206, or could be, for instance, behavioral data from a user device such as app usage, search history, etc.

Other training datasets such as nutritional training data 208, food item image training data 210, or location training data 220 may be optionally used as training inputs to varying degrees during machine learning controller 104 training. Nutritional training data 208 may include multiple datasets sourced from one or more distinct databases and from one or more vendors as depicted by data sources 212 and 214. These nutritional training data may include, for instance, records of known caloric and composition values for various food items, such as menu items available at a restaurant, packaged food items, prepared food items, or recipe outcomes. Likewise, food item image training data 210 may include multiple datasets sources from one or more distinct databases and from one or more vendors as depicted at 216 and 218. These food item image training data 210 may include, for instance, labeled images of food items. Similarly, location training data 220 may include multiple datasets sources from one or more distinct databases and from one or more vendors as depicted at 222 and 224. These location training data may include, for instance, GPS locations of restaurants, groceries stores and other locations where food items can be acquired.

Also shown in connection with FIG. 2 is an exemplary predictive model 114 that may result from an application of the training methods described herein. In some embodiments—given known production data for a particular user (e.g., 116 in FIG. 1)—the model 114 can predict a nutritional profile 226, which indicates the food item class or profile that the user is likely to have consumed. The model 114 can also be provided to include a confidence interval 228, or the likelihood that the subject of the production data 116 falls into the particularly identified nutritional profile 226. Confidence level generally refers to the specified probability of containing the parameter of the sample data on which it is based, which is the only information available about the value of the parameter. For example, if a 95% confidence level is selected, then it would mean that if the same population is sampled on numerous occasions and confidence interval estimates are made on each occasion, the resulting intervals would bracket the true population parameter in approximately 95% of the cases. An example of confidence level estimation that can be adapted for use by predictive modeling system 100 is described by G. Papadopoulos et al., “Confidence Estimation Methods for Neural Networks: A Practical Comparison,” ESANN 2000 proceedings—European Symposium on Artificial Neural Networks Bruges (Belgium), 26-8 Apr. 2000 D-Facto public., ISBN 2-930307-00-5, pp. 75-80, which is hereby incorporated by reference in its entirety. The disclosed method is just an example and is not intended to be limiting.

In some embodiments, the systems and methods described herein are embodied in a machine learning controller that—once trained—can be used to make predictions based on partial information inputs and predict outcomes in the absence of food item images or nutritional intake logs. In an exemplary embodiment, the model 114 maps a set of production data 116 for a user to a nutrition classifier or profile 226.

In some embodiments, multiple layers of the predictive models may be utilized such that the training dataset 106 might be configured to include derived training datasets as well. By way of example, refined datasets could be created that integrate location information with known restaurant locations, available menu items and consumption patterns to predict food items likely to be consumed at a particular location. Data feeds can come directly from restaurants themselves or be scraped via available web data.

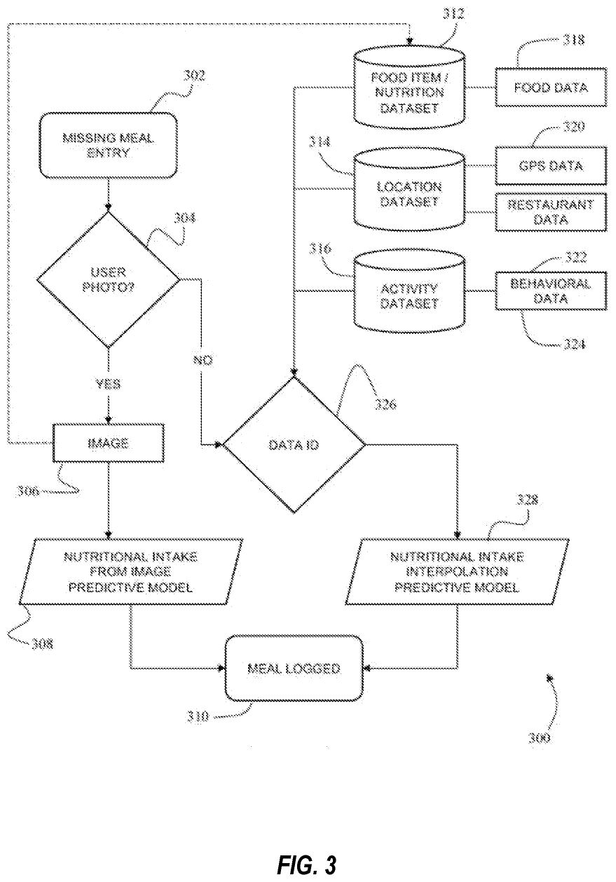

In one embodiment, an exemplary method is provided to interpolate missing food consumption entries into a nutritional intake log over a period of time. Turning to FIG. 3, one such embodiment is illustrated in method 300. A prospective missing meal entry is identified at 302. In some embodiments, the method may optionally search or query at 304 for a user photo of any food items taken around the gap in which the prospective missing meal entry is located. If one or more such photos are located at 306, then the model is used to predict nutritional intake from the image by classification of the food item and its associated nutritional values, as at 308, then the meal is logged in the nutritional intake log at 310. In some cases, it may be desirable for a feedback mechanism to store the one or more images located at 306 and associated identification at 308 in the food item and nutrition database 312.

Other databases or datasets that may be employed are location datasets 314 and activity datasets 316. Food item and nutrition datasets 312 can store data and inferences related to food items 318, such as food items images, identifications, nutritional values such as calories and composition, and the like. Location datasets 314 can store things such as GPS location data for food acquisition locations 320 and associated restaurant data 322 such as menu items, consumption patterns and the like. Activity datasets 316 can contain behavioral data 324 from, for instance, the user by way of mobile device, such as search and order histories, app usage, user location data, and the like.

If no suitable food item images were detected at 304, relevant data are identified at 326 for use in the nutritional intake interpolation predictive model at 328. The interpolation function 328 uses the food item and nutrition dataset 312, location dataset 314, and activity dataset 316 to identify the most likely food item consumption patterns for the particular time identified at 302, and the meal and nutritional characteristics so identified are logged at 310.

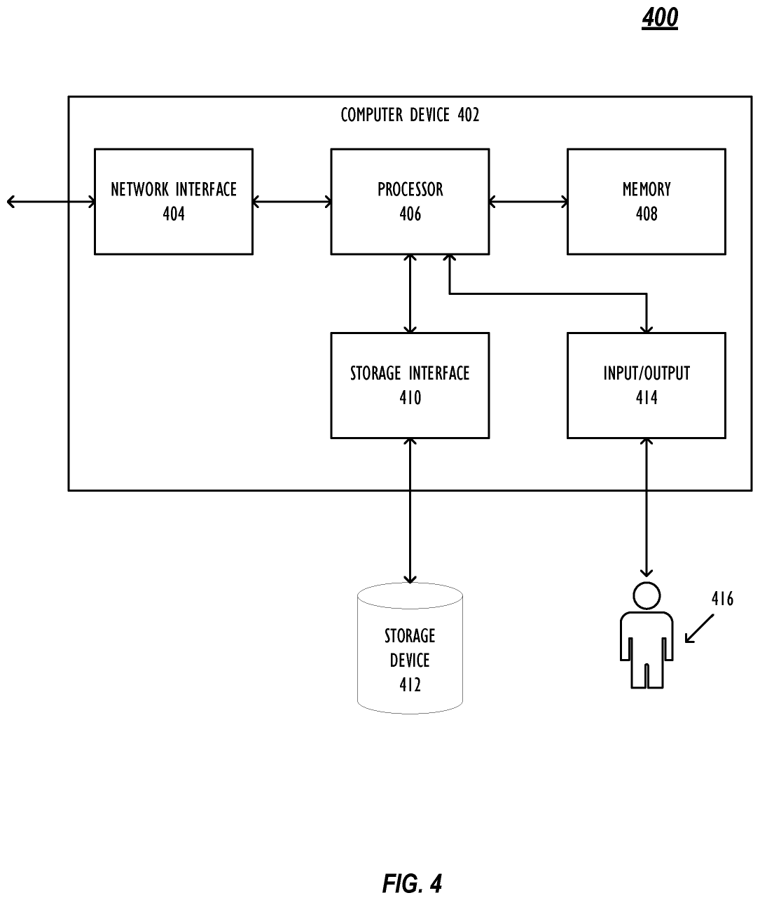

FIG. 4 depicts an exemplary configuration 400 of a computing device 402, in accordance with one embodiment of the present disclosure. Computing device 402 may be operated by a user 416. Computing device 402 may include a processor 406 for executing instructions. In some embodiments, executable instructions may be stored in a memory 408. Processor 406 may include one or more processing units (e.g., in a multi-core configuration). Memory 408 may be any device allowing information such as executable instructions and/or transaction data to be stored and retrieved. Memory 408 may include one or more computer-readable media.

Computing device 402 may also include an input/output component 414 for presenting information to user 416. Input/output component 414 may be any component capable of conveying information to user 416. In some embodiments, input/output component 414 may include an output adapter (not shown) such as a video adapter and/or an audio adapter. An output adapter may be operatively coupled to processor 406 and operatively able to be coupled to an output device such as a display device (e.g., a cathode ray tube (CRT), liquid crystal display (LCD), light emitting diode (LED) display, or “electronic ink” display) or an audio output device (e.g., a speaker or headphones).

In some embodiments, input/output component 414 may be configured to present a graphical user interface (e.g., a web browser and/or a client application) to user 416. In some embodiments, input/output component 414 may include an input device for receiving input from user 416.

The input device may include, for example, a keyboard, a pointing device, a mouse, a stylus, a touch sensitive panel (e.g., a touch pad or a touch screen), a gyroscope, an accelerometer, a position detector (e.g., a GPS sensor), a biometric input device, and/or an audio input device. A single component such as a touch screen may function as both an output device and an input device.

Computing device 402 may also include a network interface 404, communicatively coupled to a remote device such as another computing device, a network server, a storage device, cloud storage, or the like. Network interface 404 may include, for example, a wired or wireless network adapter and/or a wireless data transceiver for use with a mobile telecommunications network. Network interface 404 may receive input from user device via network, such as the internet.

Stored in memory 408 are, for example, computer-readable instructions for providing a user interface to user 416 for receiving and processing from input/output component 414. A user interface may include, among other possibilities, a web browser and/or a client application. Web browsers enable users, such as user 416, to display and interact with media and other information typically embedded on a web page or a website. A client application may allow user 416 to interact with, for example, another computing device. For example, instructions may be stored by a cloud service, and the output of the execution of the instructions may be sent to the input/output component 414.

In some embodiments the computing device 402 may include input/output components for capturing and generating data. Input/output components may include, but are not limited to, a GPS device, an accelerometer and a gyroscope for capturing geographic coordinate data or location data. Additionally, or alternatively, the input/output components may include an image sensor, such as a camera, for capturing image data. Contextual information may be determined based on the captured location data and image data, such as type of meal, time of day, or the like. Captured data and contextual information may be stored in a secure location, such as storage device 412. In some embodiments, storage device 412 may be a local storage device. Alternatively, storage device 412 may be a remote storage location, such as a cloud storage device.

Storage device 412 may be any computer-operated hardware suitable for storing and/or retrieving data, such as, but not limited to, data associated with a database. In some embodiments, storage device 412 may be integrated in computing device 402. For example, computing device 402 may include one or more hard disk drives as storage device 412.

In other embodiments, storage device 412 may be external to computing device 402 and may be accessed by a plurality of computing devices 402. For example, storage device 412 may include a storage area network (SAN), a network attached storage (NAS) system, and/or multiple storage units such as hard disks and/or solid-state disks in a redundant array of inexpensive disks (RAID) configuration.

In some embodiments, processor 406 may be operatively coupled to storage device 412 via a storage interface 410. Storage interface 410 may be any component capable of providing processor 406 with access to storage device 412. Storage interface 410 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 406 with access to storage device 412.

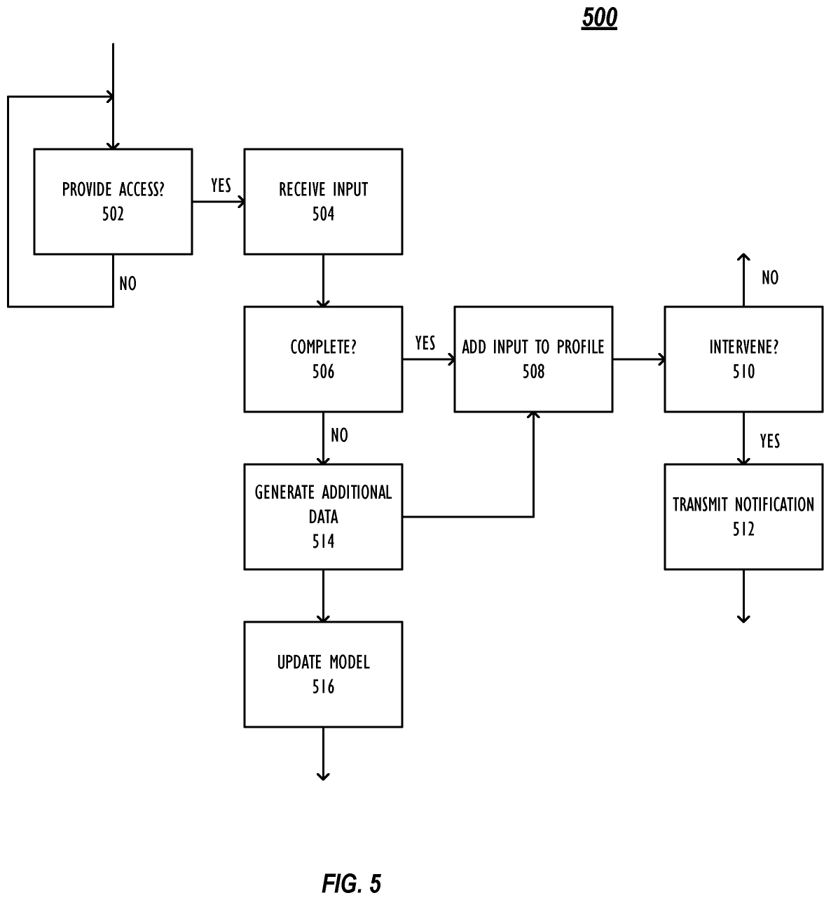

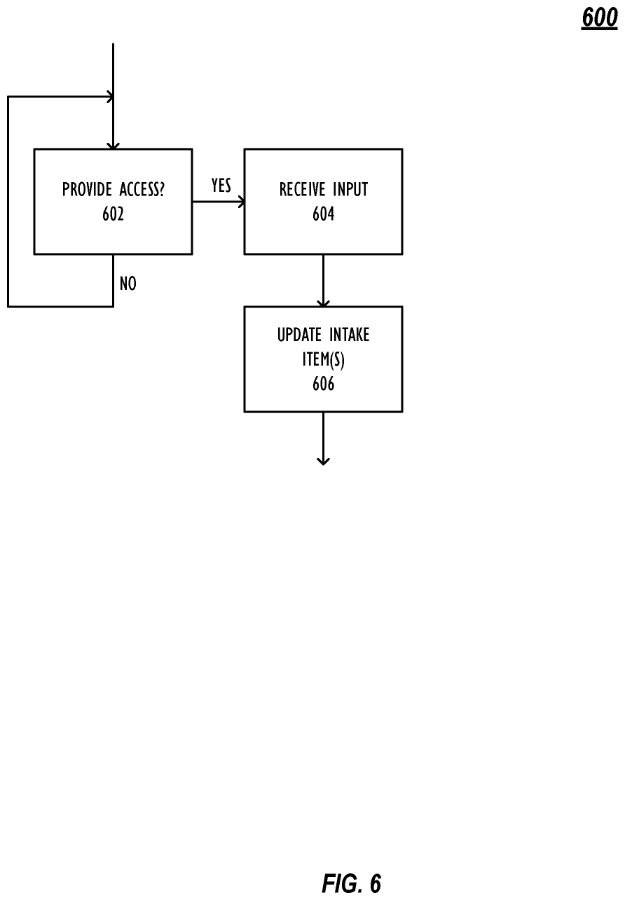

Processor 406 may execute computer-executable instructions for implementing aspects of the disclosure. In some embodiments, the processor 406 may be transformed into a special purpose microprocessor by executing computer-executable instructions or by otherwise being programmed. For example, the processor 406 may be programmed with the instruction such as illustrated in FIGS. 5 and 6.

FIG. 5 depicts an exemplary method 500 for providing a nutritional intake modeling (NIM) platform. Method 500 may be implemented by a computing device, such as computing device 402 shown in FIG. 4. Further, steps of method 500 may be performed in conjunction with a plurality of client computing devices (e.g., patient devices), provider computing devices (e.g., dietitian devices, nutrition planner devices), server devices, mobile platform provider devices, or the like.

Method 500 may include providing 502 access to the NIM platform. Access requests may be received from new and existing platform users. Platform users may include, but are not limited to, patients, healthcare providers, dietitians, administrative users, data validators, developers, or the like. Access may be requested via, for example, a mobile application on a user's device. A user may be prompted to provide user credentials (e.g., username/password). Additionally, the user may be prompted to provide additional credentials, such as multi-factor authentication credentials (e.g., security pin, RSA, etc.). If access is denied (NO), the user may be shown a message to try again and a user login screen. If the user is found to be authorized (YES), the user is provided access to the platform. In some embodiments, the user may be provided the opportunity to create a new account with the platform.

Method 500 may include receiving 504 input from the user. In this example, the user is one that uses the platform to track, log and record food items they consume. Input may be provided in text, image, video, and/or audio form. Input may also include the name of the item, ingredients, food group, category, or the like. Additional data may be provided, such as time of day, location, restaurant, or the like. This information may be provided directly by the user or indirectly from components of the user's device. For example, the user's device may include a GPS receiver that determines the user's location and adds to the input automatically. Other information may be gleaned from the user's location data, such as the name of restaurant where the intake item was consumed, time of day, or the like.

Method 500 may include determining 506 whether the user's input is complete. For example, when the user inputs a food intake item, some information about the food intake item may be determined to be missing. If the food intake item is considered complete (YES) and no information is determined to be missing, the process moves to step 508. In step 508, the input, or intake item, may be added to the user's health record, or food log, in the user's profile. Based on the user's goals, an intervention 510 step may be needed. For example, the user may set short-term and long-term goals regarding their nutritional intake (e.g., proteins, carbs, fats, vegetables, fruits, dairy). Real-time intervention may be based on the user's information as well as information provided from other users, previously captured data, global data from all users, and individual patient goals (e.g., current and future). If an intervention is needed (YES), the method may transmit 512 a notification to the user. For example, the user may be notified via, for example, an in-app message, a popup message, a text message, email, or a phone call. Additionally, or alternatively, another user may receive the notification, such as a healthcare professional, an accountability partner, or a dietitian associated with the user. The notification may contain a note indicating that a goal is being reached or has been achieved. Alternatively, the notification may serve as a reminder to the user about their goals to help them stay on track. For example, the notification may inform the user that they are consuming above or below their average number of calories and/or protein. A subsequent notification may be sent to the user at their next meal to help them stay on track. For example, if the user was above or below their average at breakfast, a notification may be sent to the user to consume less (if above average) or more (if below average) at lunch time. An additional notification may be provided, for example, at dinner time based on their consumption through out the day. In some embodiments, a notification may be generated to provide suggestions to the user. For example, based on the user's prior intake, the model may provide suggestions to the user to help them stay on track with their goals. In this example, meals may be suggested to the user for future consumption. Suggestions over a period of time may be provided, such as over the next few days or the next week. Daily suggestions may be provided. For example, based on the user's intake at breakfast, the components of a dinner may be provided to the user to assist them with staying within their goals.

Method 500 may include determining 506 that information about the intake item is incomplete (NO). In this example, method 500 may include generating 514 additional data about an intake item. In this example, data inputs provided by the user may be provided to a predictive model (e.g., model 114). Information may be predicted using the model to provide nutritional information (e.g., calories, protein), food type/classification, or the like, as described herein and above. In some embodiments, the predictive model may be continuously updated 516 using the additional data predicted. Additionally, users of the system, such as patients and providers, may validate the user updated data and predicted data. For example, a dietitian may validate the identification of an item, its nutritional content (e.g., calories, protein), or the like. Once generated, the additional data may be added 508 to the user's profile and the process continues.

FIG. 6 depicts method 600 where a user, such as a dietitian or health provider may be provided 602 access to the platform. Method 600 may include receiving 604 data input from the user pertaining to one or more intake items. For example, the user may access another user's profile and update 606 one or more intake items in their profile. In one embodiment, the user may update details regarding an intake item, such as nutritional data (e.g., calories, protein). In another embodiment, the user may apply labels or tags to intake items.

The embodiments described herein may be deployed as a cloud platform, utilize mobile or edge computing, or a combination thereof. For example, some of the training systems and methods may be initially performed centrally, with user access available via one or more mobile applications in which model computing operations are carried out.

As will be appreciated based upon the foregoing specification, the above-described embodiments of the disclosure may be implemented using computer programming or engineering techniques including computer software, firmware, hardware or any combination or subset thereof. Any such resulting program, having computer-readable code, may be embodied or provided within one or more computer-readable media, thereby making a computer program product, i.e., an article of manufacture, according to the discussed embodiments of the disclosure. The computer-readable media may be, for example, but is not limited to, a fixed (hard) drive, diskette, optical disk, magnetic tape, semiconductor memory such as read-only memory (ROM), and/or any transmitting/receiving medium such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor and can be implemented in a high-level procedural and/or object-oriented programming language, and/or in assembly/machine language. As used herein, the terms “machine-readable medium” and “computer-readable medium” refer to any computer program product, apparatus and/or device (e.g., magnetic discs, optical disks, memory, or Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISCs), application specific integrated circuits (ASICs), logic circuits, and any other circuit or processor capable of executing the functions described herein. The above examples are examples only and are thus not intended to limit in any way the definition and/or meaning of the term “processor.”

As used herein, the terms “software” and “firmware” are interchangeable and include any computer program stored in memory for execution by a processor, including RAM memory, ROM memory, EPROM memory, EEPROM memory, and non-volatile RAM (NVRAM) memory. The above memory types are examples only, and are thus not limiting as to the types of memory usable for storage of a computer program.

In one embodiment, a computer program is provided, and the program is embodied on a computer-readable medium. In an example embodiment, the system is executed on a single computer system, without requiring a connection to a sever computer. In a further embodiment, the system is run on a Windows® environment (Windows is a registered trademark of Microsoft Corporation, Redmond, Wash.). In yet another embodiment, the system is run on a mainframe environment and a UNIX® server environment (UNIX is a registered trademark of X/Open Company Limited located in Reading, Berkshire, United Kingdom). The application is flexible and designed to run in various different environments without compromising any major functionality. In some embodiments, the system includes multiple components distributed among a plurality of computing devices. One or more components may be in the form of computer-executable instructions embodied in a computer-readable medium. The systems and processes are not limited to the specific embodiments described herein. In addition, components of each system and each process can be practiced independent and separate from other components and processes described herein. Each component and process can also be used in combination with other assembly packages and processes.

As used herein, an element or step recited in the singular and preceded by the word “a” or “an” should be understood as not excluding plural elements or steps, unless such exclusion is explicitly recited. Furthermore, references to “example embodiment” or “one embodiment” of the present disclosure are not intended to be interpreted as excluding the existence of additional embodiments that also incorporate the recited features.

Any embodiment of the present invention may include any of the optional or preferred features of the other embodiments of the present invention. The exemplary embodiments herein disclosed are not intended to be exhaustive or to unnecessarily limit the scope of the invention. The exemplary embodiments were chosen and described in order to explain the principles of the present invention so that others skilled in the art may practice the invention. Having shown and described exemplary embodiments of the present invention, those skilled in the art will realize that many variations and modifications may be made to the described invention. Many of those variations and modifications will provide the same result and fall within the spirit of the claimed invention. It is the intention, therefore, to limit the invention only as indicated by the scope of the claims.

1. A nutritional intake modeling (NIM) computing device for a nutrition platform, accessible by a plurality of users and a plurality of providers, comprising of one or more processors which are configured to:
provide access to the nutrition platform to a plurality of user devices associated with the plurality of users;
receive, from at least one user of the plurality of users, data input captured by the at least one user's user device;
generate, using a predictive model, an intake item based on a profile of the at least one user and the received data input;
add the intake item to the user's profile; and
store the user's profile on a storage device communicatively coupled to the NIM computing device.

2. The NIM computing device of claim 1, wherein the one or more processors are further configured to:
provide access to the nutrition platform to a plurality of provider devices associated with the plurality of providers;
assign at least one provider of the plurality of providers to the at least one user; and
provide access to the at least one user's profile to the at least one provider.

3. The NIM computing device of claim 2, wherein the one or more processors are further configured to:
receive, from the at least one provider, one or more tags for the intake item; and
update the intake item to include the one or more tags.

4. The NIM computing device of claim 1, wherein the plurality of providers are registered dietitians.

5. The NIM computing device of claim 1, wherein the one or more processors are further configured to:
retrieve additional information based on the intake item; and
update the intake item to include the additional information.

6. The NIM computing device of claim 5, wherein the additional information includes one or more of ingredients, food groups, and food components.

7. The NIM computing device of claim 5, wherein the additional information is retrieved using an internal nutritional algorithm based on one or more properties of the intake item.

8. The NIM computing device of claim 1, wherein the predictive model is created using one or more training datasets using artificial intelligence, machine learning, or a combination thereof.

9. The NIM computing device of claim 8, wherein the one or more training datasets are validated by one or more of the plurality of users.

10. The NIM computing device of claim 1, wherein the one or more processors are further configured to:
update the predictive model based on the received data input, the generated intake item, and, at least in part, the user's profile.

11. A non-transitory computer-readable medium configured to store instructions that, when executed by a processor, implement:
creating a predictive model using a plurality of training datasets;
providing access to a nutrition platform to a plurality of user devices associated with a plurality of users;
providing access to the nutrition platform to a plurality of provider devices associated with a plurality of providers;
receiving, from a user of the plurality of users, data input captured by the user's device;
generating, using the predictive model, an intake item based on a profile of the user and the received data input;
adding the intake item to the user's profile; and
storing the user's profile on a storage device for subsequent retrieval.

12. The non-transitory computer-readable medium of claim 11, wherein the instructions further implement:
assigning a provider of the plurality of providers to the user; and
providing access to the user's profile to the provider.

13. The non-transitory computer-readable medium of claim 11, wherein creating the predictive model includes:
receiving the plurality of training datasets from a plurality of sources;
validating one or more of the plurality of training datasets based on input received from one or more of the plurality of providers; and
creating the predictive model by correlating data of the training datasets.

14. The non-transitory computer-readable medium of claim 13, wherein the one or more of the plurality of training datasets are validated by one or more of the plurality of providers.

15. The non-transitory computer-readable medium of claim 13, wherein the predictive model is updated by correlating the data input and the generated intake item.

16. A method, comprising:
receiving data input captured by a client device associated with a patient;
generating, using a predictive model, an intake item based on the received data input;
updating a record associated with the patient to include the intake item;
generating an alert based, at least in part, on at least one parameter stored in the record and the intake item; and
sending the alert to the client device.

17. The method of claim 16, wherein the data input is captured by a smartphone.

18. The method of claim 17, wherein the data input is captured by a camera of the smartphone.

19. The method of claim 16, wherein the at least one parameter is either a goal set by the patient or a goal set by a provider associated with the patient.

20. The method of claim 16, further comprising:
receiving additional information for the intake item from one or more of a plurality of providers; and
updating the intake item to include the additional information.
